(19) United States
(12) Patent Application Publication
(10) Pub. No.: US 2022/0047626 A1
(43) Pub. Date: Feb. 17, 2022

(54) COMPOSITION OF A SPRAY FORMULA TO CONTROL MASTITIS IN BOVINES

(71) Applicant: COPPER ANDINO S.A., Providencia, Santiago (CL)

(72) Inventor: Luis Alberto AMESTICA SALAZAR,, Providencia, Santiago (CL)

(21) Appl. No.: 17/416,054

(22) PCT Filed: Dec. 18, 2018
(86) PCT No.: PCT/CL2018/050128

§ 371 Date: Jun. 18, 2021

(51) Int. Cl. A61K 33/30; A61K 31/315; A61K 31/555; A61K 45/06; A61K 9/08; A61P 31/04

(52) U.S. Cl. CPC A61K 33/30; A61K 31/315; A61P 31/04; A61K 45/06; A61K 9/08; A61K 31/555

(57) ABSTRACT

The present invention corresponds to the composition of a unique formula to be used as pre-dipping and post-dipping in robot milking in order to control mastitis in bovines. The composition comprises at least one organic zinc salt, water soluble and with biocidal properties, where the concentration of zinc ions is in the range of 5,000 to 60,000 ppm. The salts with biocidal properties were selected for having a functional group similar to proteins. Organic zinc salts are degradable, less irritating to the cow's teat, improve the skin condition, have an anti-inflammatory effect, accelerate wound healing processes and eliminate bacteria and fungi. The organic zinc salts selected for this invention were the following: gluconate, lactate, glycinate, lysinate, citrate trihydrate, picolinate, and acetate. Zinc sulfate monohydrate can be optionally added, but in low concentration, less than 1.5% of the final product. The viscosity of the spray composition should be in the range of 3 to 5 centipoise (cP), at room temperature. Finally, and very importantly, the composition has no corrosive activity on metals and non-metals, such as stainless steel, carbon steel, computer cards and other components found in bovine milking robots.

### TECHNICAL FIELD

The present invention is related to a biocidal composition based on organic zinc salts for use in the control of infections in the mucous membrane in mammals, particularly to prevent and treat mastitis in bovines.

The invention provides a low-viscosity, non-corrosive composition to be applied in a spray form as pre-dipping and/or post-dipping in the milking process using robots, with excellent disinfectant performance, capable of eliminating or reducing mastitis caused by bacteria and fungi, and where the composition is also non-irritating, improves skin conditions, has an anti-inflammatory effect, accelerates wound healing and eliminates bacteria and fungi.

### BACKGROUND OF THE INVENTION

Clinical and subclinical mastitis is described as inflammation of the mammary gland in cattle due to infection of the mucous membrane of the udders caused by bacteria, fungi, yeast and other pathogenic microorganisms, which enter in the udder duct infecting one or more mammary quarters during the lactation period of bovines. This infection causes inflammation of the udder and must be treated with antibiotics in order to eliminate the pathogen and restore the functionality of the organ. Clinical mastitis has an enormous cost for milk producers due to an alteration in quality (it cannot be sold) and production decreases. Additionally, there is a high cost associated with the treatment, which can take more than a week to eliminate the infection, even more when the treatment is not adequate, the cow could die. In the case of subclinical mastitis, the clinical signs in the udders are invisible and can only be detected by a decrease in milk production, an increase in the Somatic Cell Count (SCC), and the number of Colony Forming Units. (UFC). These changes have an impact on the quality and therefore on the price of milk.

The most common bacteria that can cause clinical and subclinical mastitis, both related to a high somatic cell count, are: Escherichia coli, Staphylococcus aureus, Streptococcus uberis, Streptococcus agalactiae, Streptococcus dysgalactiae, Corynebacterium spp., Staphylococcus coagulasa negativa, Bacillus spp., Nocardia spp., y Pseudomonas spp. among others. Some of these bacteria are part of the normal microbiota of the skin, however they can eventually cause mastitis when they enter the breast quarter. In addition to bacteria, it has been found that the presence of fungi, yeast and mycoplasma can aggravate mastitis. Furthermore, mycoplasma is a small, highly contagious bacteria for which there is no effective treatment today and which requires separating the cow from the herd before the infection spreads.

Once the milking process is complete, the udder sphincter closes in order to protect the udder canal against the entry of any bacteria or other pathogenic microorganisms. The sphincter muscle takes between 20 and 30 minutes to close completely and the udder canal remains dilated for up to 2 hours. It is during this period of time that the risk of entry of bacteria or other pathogens is critical, which is why the dairy industry has developed biocidal compositions (dipping) to prevent the entry of microorganisms, which are applied after the milking process. (post-dipping). Additionally, it is common practice in the dairy industry to wash the udders before milking with a disinfectant solution, called pre-dipping.

It is usual that the solution used as pre-dipping, to clean the udders before the milking process, has less biocidal power than the solution applied to the udders after milking, post-dipping. In most dairy milking using the carousel system, the pre-dipping solution is of low viscosity, similar to that of water, while the post-dipping is of high viscosity (such as a cream, gel or viscous liquids). The chemical composition of the pre and post dipping are completely different, and so is their antimicrobial or biocidal performance. The former is mainly used to clean the udder before milking and post-dipping is used after milking to seal the udder duct in order to avoid infection.

In recent years, milking by robots has become a standard procedure in large dairies. There are several brands of milking robots. Some of them use the same biocidal solution as pre and post dipping, due to their configuration. In robotic milking, the risk of infections increases, since all cows in the herd use the same teat cups for milking This condition makes it necessary to have a dipping with greater biocidal power. The robot sprays the dipping on the udders for a period of between 4 to 6 seconds.

The composition of the dipping used in carousel-type milking, by immersion, is not suitable to be used in a robotic system, because the viscosity is very high, reaching in most cases values over 1,000 centipoise (cP), such as the case of creams and gels, both of which are difficult to spray.

On the other hand, if the viscosity of the dipping is close to that of water (1 cP), the product is atomized, forming a circular ring, which does not fully and homogeneously moisten the udders. From the above, it follows that the dipping characteristics must be very special and very few of the products on the market meet this need. In fact, in our experience, this is one of the reasons why the incidence of mastitis is higher in robotic milking systems than in carousel-type systems.

The dipping agents that are currently used to control mastitis incorporate different biocidal agents in their composition, among these, one of the most used is iodine, which acts as an oxidizing agent for the vital cellular components of bacteria, generating precipitation of proteins in microorganisms and cell death. Iodine in solution is marketed by various dipping manufacturers with total iodine concentrations that may vary between 0.25% and 2%. The 0.25% solution in iodine has a very low biocidal property and a viscosity close to 4 cP, as the iodine concentration increases its activity, the viscosity increases. This is why a dipping based on iodine with a concentration of 0.5% or higher is not suitable for atomization by robots, since it is very viscous. Additionally, another important disadvantage of iodine-based dipping is that small amounts of this element pass into the milk, increasing the iodine concentration in it, this is considered a problem in many countries, because it has been associated with an increase in hyperthyroidism in milk consumers.

Chlorhexidine is another biocidal chemical compound used as an active agent in dipping, which acts by destabilizing and penetrating the membranes of bacterial cells, destroying them and causing them to die. However, it has been seen that although this compound is an excellent antimicrobial, it brings several risks on the udders and cows due to the chemical bonds between this compound and keratin, present in the soft and hard tissues of the cow, causing irritation and producing alterations in the sense of taste of the cow and is toxic to the tympanic membrane and the cornea. At high concentrations it produces tissue necrosis and hypersensitivity reactions. The biocidal capacity of chlorhexidine can be deactivated by the presence of organic material, such as blood, pus, and necrotic tissue. Finally, Staphylococcus strains can develop resistance mechanisms to this compound.

Acidified sodium chloride solutions can also be used as biocidal agents in dipping, where the mechanism of action is similar to that of iodine (oxidant). Being effective in eliminating bacteria, yeasts, fungi and viruses. But one of its limitations is that it has a short active life.

There are dipping formulations that use peroxides, peracids, and organic acids as active biocidal agents. These compounds can be used separately or in combination. Lately, the trend is to use them in combination to enhance their effectiveness, while peroxides and peracids are oxidizing agents, organic acids act by destroying the cell membrane and deactivating some essential enzymes in the bacteria. Some of the organic acids used are salicylic acid, caprylic acid, glutamic acid, heptanoic acid, dodecylbenzene acid, and lactic acid. In some cases the acids need an activating agent to exert their biocidal power, in this case the mixture of the products must be used immediately, since it is deactivated over time, making its application complex and impractical in the industry.

Other active compounds used in dipping formulations are metal salts: such as copper salts, mainly copper sulfate pentahydrate, and also copper salts combined with zinc salts. The most frequently mentioned zinc salt is zinc sulfate mono or heptahydrate. For example, Gonzalez in U.S. Pat. No. 9,936,705 B2 proposes the use of a gel that has as a biocidal active agent a mixture of copper sulfate and zinc sulfate, to be used as post dipping in the prevention of mastitis. Copper sulfate pentahydrate and zinc sulfate heptahydrate are in the range of 0.01% to 10% by weight. The gel form is achieved by adding large amounts of polyvinyl alcohol (PVA) (in the range of 3%-10%) and xanthan gum (in the range of 0.2-2%). The formula has emollients and humectants in the 5-30% range. The manufacturing process of this gel is complex and requires a process temperature of over 60° C. and stirring for 2.5 hours, to avoid the formation of lumps. The resulting gel is used as a dipping where the udders are dipped after milking The product (3% PVA and 0.2% xanthic gum) is a highly viscous gel that cannot be sprayed onto the udders.

The incorporation of copper ions as a biocidal agent in aqueous dipping formulations, solutions, creams or gels, cause corrosion in the metal parts of both the robot and the metal infrastructure of the milking stations. The corrosive effect of copper ions is well documented in the literature, corrosion occurs even with very low concentrations of copper ions, few parts per million, as described in “The role of solution chemistry on the corrosion of copper in tap water: the effect of dissolved silica on uniform in localized attack”, Corrosion Science and Technology, Volume 47, Issue 10, Octubre 1996, pages 559-567. Corrosion induced by the presence of copper ions in electronic equipment and computer cards is also well documented, “Corrosion in electronics: Overview of failures and countermeasures”, University of Denmark, Jellesen Marten et al, published in Proceedings of EuroCorr 2014. Therefore, the use of copper salts in dipping formulations to be used by robots is not a viable option.

Patent application EP 2724724 A1 by Carly Vulders et al. describes the use of an atomizable solution using copper and zinc chelating agents (EDTA copper and zinc salts) for the treatment and prevention of epithelial infections in animals: goats, sheep, equines and cattle. The referenced solution contains 50-60% alcohol to which micronized copper and zinc chelates are added in the 5-50% range and with a water content of less than 20%. The ratio between chelates of copper and zinc is in the range of 1.2 and 2.1. Alcohol is used as a solvent because it evaporates quickly, which allows micronized copper and zinc to be left on the skin. This formula helps to reduce or eliminate microorganisms that cause infections in the dermis, epidermis and hooves. One of the main problems with using this formulation in robot milking systems is its corrosiveness.

In 2001 U.S. Pat. No. 6,183,785 B1, Geoffrey J. Westfall describes a formulation and method for preventing mastitis in lactating animals In particular, the formula involves a mixture of a zinc salt, preferably gluconate, and chlorhexidine. The solution is applied to the udders, for example cow udders, by spraying or dipping the udders in the solution. The zinc salt is added in the range of 0.1 to 5%, while the chlorhexidine is present in the range of 0.1% to 4%. The solvent includes a mixture of water and alcohol, the latter is preferably selected from alcohols with less than 3 carbon atoms. The formula cannot be used in robot milking as aqueous chlorhexidine solutions are known to corrode stainless steel. (International Endodontic Journal 2002, August 35(8): 655-9).

The same author, Geoffrey J. Westfall, in patent US 2006/009477 A1, presents a new and improved version of his earlier patent. The formulation compromises zinc EDTA and chlorhexidine. The formulation has 0.05% to 5% of zinc EDTA and 0.05% to 5% of chlorhexidine. The zinc EDTA and chlorhexidine ratio is 1:1. The carrier is preferably water and aerosol propellant According to the author, the aerosol has a chilling effect on the udders, which causes the sphincter muscle and the udder orifice to contract, which would act to prevent bacteria from entering the duct causing infections.

Manuel Jauregui Renault in patent application WO9913892 A1, “Antimastitic Pharmaceutical Composition” presents a formulation based on oil extracted from plants with the addition of zinc sulfate to treat mastitis in bovine, caprine and ovine animals The formula also contains EDTA, citric acid, ascorbic acid and sodium benzoate. The oil can be Aloe vera, Agave atrovirens, Citrus lemon, Melaleuca alternifolia and Symphytum officinale. The concentration of zinc sulfate salts in the formula is in the 0.002%-0.003% range. Zinc salts are not the active ingredient in the formulation, it is incorporated to stimulate an immune response. It does not indicate viscosity or form of application of this formula on animals

### SUMMARY OF THE INVENTION

The present invention is related to compositions or formulations of a pre or post milking dipping to control mastitis in cattle, increasing the protection offered by the current formulations present on the market in spray form for robot milking systems.

Among other properties of the composition according to the present invention for controlling the mastitis are that it: is not irritant for cattle skin, improves udder skin conditions, has an anti-inflammatory effect, increases the rate of wound healing, eliminates over 99.99% of bacteria in less than 30 seconds of contact time, and does not corrode metals.

Additionally, another objective of the composition due to its characteristics is to provide a formulation that can be used as a single product, pre and post dipping, for use in milking robots, having excellent cleaning and disinfecting properties.

The composition is an aqueous well-balanced formula that incorporates auxiliary elements, comprising:

a) Organic zinc salts with biocide properties,

b) humectant agents suitable for foods,

c) viscosity control additives such that, when combined with the humectant agents allow the achievement of a viscosity target between 3-5 cP, and

d) water to form an emulsion.

Biocidal salts are selected from zinc salts, preferably water-soluble organic salts, in which the organic part of the salt has a configuration that is similar to a protein. The similarity of the anionic part of the salt to a protein ensures its compatibility with the skin and confers greater safety of use. It was found that, using the adequate concentration of zinc salts as a biocidal active ingredient in the dipping formulation, excellent performance as a disinfectant is obtained and mastitis can be reduced or eliminated There is no need to add another antimicrobial agent, such as chlorhexidine or copper sulfate, avoiding problems such as robot corrosion, udder irritation or toxicity.

Zinc salts do not irritate the udders and improve the skin condition. In addition, they have an anti-inflammatory effect, they accelerate wound healing and act as biocides, eliminating bacteria and fungi.

Thus, it has been found that the use of zinc salts, preferably organic added in the appropriate concentration in a well-balanced formula, can provide a better antimicrobial performance in the prevention and elimination of mastitis than those formulas that incorporate iodides, mixtures of copper-zinc salts, chlorhexidine-zinc salts, and peracids-glutamic acid. The composition according to the present invention has a better performance in the elimination of bacteria isolated in herds of animals that cause clinical and subclinical mastitis, being confirmed in laboratory tests.

The most desirable organic zinc salts are zinc gluconate, zinc glycinate, zinc lactate, zinc citrate trihydrate, zinc picolinate, zinc acetate, and zinc lysinate. Formulations with two or more organic zinc salts have been found to offer better biocidal activity than a single zinc salt.

The concentration of zinc ions in the dipping formula according to the present invention is in the range of 5,000 and 60,000 ppm, preferably in the range of 10,000 to 25,000 ppm. Although there are no regulations on the zinc content in milk, the amount of zinc that passes into the milk was measured, after its use as pre and post dipping in milking using a robot, finding that the total concentration of zinc in the milk increases by less than 5% versus its natural content, when zinc ion dippings are not used.

It was concluded that inorganic zinc salts, such as zinc chloride and zinc nitrate are undesirable for this formulation due to the corrosive potential on metals of the anionic part. However, it was found that zinc sulfate can optionally be added to the formulation as a suitable inorganic salt in combination with at least one organic zinc salt.

Zinc sulfate monohydrate can be used but in a concentration that must be below 1.5%, preferably less than 1.1% (which is equivalent to 4,000 ppm of zinc ion) in the present invention. In the experimentation it was found that over 10,000 ppm of zinc ion from zinc sulfate monohydrate in the dipping (equivalent to 2.74% of zinc sulfate monohydrate in formula) causes a deterioration in the stability of the formula, since with the time, the polymerization of polyvinyl alcohol (PVA) occurs, creating an insoluble gel that sediments, clogging the robot's spray systems.

Test of microbiological challenges with strains of Escherichia coli, Staphylococcus aureus, Streptococcus uberis, Streptococcus agalactiae, Streptococcus dysgalactiae, Corynebacterium spp., Staphylococcus coagulase negative, Bacillus spp., Nocardia spp., Pseudomonas spp. isolated from a herd of cows show that the formula of the present invention kills over 99.99% of bacteria in 30 seconds of contact. The time required to kill bacteria is critical since it is the time immediately after milking that the udder duct remains open and exposed to entry of infection-causing microorganisms.

### DETAILED DESCRIPTION OF THE INVENTION

The present invention refers to a composition to control mastitis that can be used in robot milking because it is not corrosive and has a viscosity suitable for being sprayed.

Composition

The composition is an aqueous well-balanced formula that incorporates auxiliary elements, comprising:

a) the biocide organic zinc salts, selected for having a functional group similar to proteins with biocide properties, selected from the group consisting of zinc gluconate, zinc glycinate, zinc lactate, zinc citrate trihydrate, zinc picolinate, zinc acetate, and zinc lysine; the concentration of the zinc salts being in the range of 5,000 to 60,000 ppm;

b) humectant agents suitable for foods, selected from the group consisting of glycerin, propylene glycol, hexylene glycol, butylene glycol, glyceryl triacetate, aloe vera, pyrrolidone carboxylic acid, sorbitol, sodium lactate, panthenol, among others. The preferred humectant is glycerin since it has one of the best moisture retention per weight of product ratio. In addition, glycerin has a higher viscosity (950 cP), compared with sorbitol (200 cP) and propylene glycol (52 cP), that allows for better management of the viscosity in the final product. The concentration of humectant agents in the finished product should in the range of 5-20% by weight. The most suitable humectant agents are a mixture of glycerin and propylene glycol in a weight ratio in the range of 6 to 1 to 15 to 1, as glycerin has the highest viscosity and propylene glycol creates foam, so the amount has to be restricted to the described ratio;

c) Viscosity control additives (viscosifier), such that in combination with the humectant ingredient used achieves the target viscosity in the 3-5 centipoise range, where one of the preferred additives is polyvinyl alcohol (PVA). The amount of PVA to achieve the desired viscosity is manageable (2-4% by weight), the fluid is Newtonian, does not cause phase separation and does not need to use heat or heating in the process of preparing the composition. Other thickeners with a very small amount can make the fluid of the composition (emulsion in water) become a non-Newtonian fluid with a much higher viscosity than the target, this is the case with algic acid, agar, carrageenan, pectin, gelatin, and xanthan gum. The use of these thickeners also requires a hot process to avoid lumps and achieve a stable phase over time. To reach a viscosity of 3-5 cP, these thickeners should be added in small amounts (0.1-0.3%); where solutions with these amounts are not stable over time, and the thickener tends to precipitate and separate from the solution, producing phase separation; and

d) water to form an emulsion (c.s.p. 100%)

Optionally, as previously mentioned, the suitable inorganic salt that can be added in combination with at least one organic zinc salt is zinc sulfate monohydrate in a concentration below 1.5%, preferably below 1.1% by weight.

Optionally, other agents could be added, such as colorants, perfume, vitamins, alpha-hydroxy-acids, beta hydroxy-acids, hydroquinone and other agents which aid skin treatment.

Process

The dipping solution (composition) is prepared in three stages, in the first stage the zinc salts are dissolved in water. In the second stage, the organic material (humectants and thickening agents) is added. Finally, the third stage involves the addition of the colorant.

The composition is prepared in a mix tank (a tank with an axial agitator).

The main steps of the process are:
a) A mix tank is loaded with demineralised water, depending on the composition of the formula (c.s.p. 100%).
b) Zinc salts are added in the range from 6% to 20% dependent upon the selected group of salts and zinc ion concentration target and the system is agitated until complete dissolution of zinc salts. The agitation time goes from 10-20 minutes at an agitation speed of 300-400 rpm until complete dissolution is achieved.
c) Humectants are added in the range of 5-20%. The system is agitated until the formation of the emulsion is ensured, the agitation time is 10-20 minutes and the agitation speed is 300-600 rpm.
d) Thickening agent is added in the range of 2 to 4% to achieve the target viscosity.
e) Agitation is continued until a homogeneous mixture is obtained.
f) A sample is taken from the tank and visually inspected with respect to the non-presence of lumps and the viscosity is measured.
g) Finally, if everything fulfills the requirements a colorant solution is added: The colorant is previously dissolved in a small amount of water.

It is necessary to confirm that all zinc salts are completely dissolved before adding the humectants and thickening agents. If not, lumps may form.
The order of addition is critical to obtain a dipping with the required characteristics.

The selected humectant agents are glycerin and propylene glycol, where glycerin has the best water retention among all known humectants, while propylene glycol is selected because it reduces the surface tension of dipping and acts as a stabilizer.

The selected viscosifying agent is PVA. Its concentration is between 2% and 4%, preferably in the range of 3% to 4% to obtain a viscosity of 3 to 5 cP. No other thickening agent is desirable for the production of this product: such as algic acid, agar, carrageenan, pectin, gelatin, xanthan gum, and others.

The process is conducted at room temperature, which differs with most process that use a temperature of 60° C.-80° C. It has been found that an increase in the rate of agitation over 1000 rpm promotes the polymerization of PVA, causing lumps in finished product, which reduces the shelf life of the dipping.

Biocide Organic Zinc Salts
The active ingredient corresponds to organic zinc salts, where two or more salts have a higher biocidal activity than a single zinc salt in the composition. The total concentration of zinc ions resulting from the contribution of all salts should be in the range of 5,000 to 60,000 ppm, preferably in the range of 10,000 to 25,000 ppm.

Therefore, the type, amount and solubility of the selected zinc salts are relevant for the present invention in order to achieve the target zinc ion concentration with the selected salt or pairs of salts.

The selected zinc organic salts for this present innovation are: zinc gluconate, zinc glycinate, zinc lactate, zinc citrate trihydrate, zinc picolinate, zinc acetate, and zinc lysine.

The preferred mixtures of zinc salts are zinc gluconate and zinc lactate in a ratio of 5/1 to 1/5 by weight, zinc gluconate and zinc glycinate in a ratio of 5/1 to 1/5 by weight, zinc gluconate and zinc acetate in a ratio of 5/1 to 1/3 by weight. In addition the mixtures of zinc glycinate and zinc lactate in a ratio 3/1 to 1/3 by weight, zinc lactate and zinc acetate in a ratio of 5/1 to 1/5 by weight can be also used. In all these mixtures, a third zinc salt such as zinc citrate trihydrate and zinc picolinate could be added in a concentration of 1% and 0.5% by weight, respectively in the final product.

The use of 2 or more zinc salts in the mixture has proven to increase the antimicrobial performance. In addition, it provides flexibility to achieve the target zinc ion concentration in finished product given the water solubility of these salts.

Zinc Sulfate

Optionally, zinc sulfate mono-hydrate can be added in combination with at least one organic zinc salt in order to achieve a proper concentration of zinc ions. Zinc sulfate mono-hydrate is added in such a way that its concentration should be below 1.5%. The reasons for this restriction is that concentrations above that value tend to react with PVA, forming lumps. The interaction is due to the reaction within the sulfate from the zinc sulfate with the PVA.

The salt mixtures that are preferred to be mixed with zinc sulfate mono hydrate are zinc gluconate and zinc lactate; zinc gluconate and zinc glycinate; zinc gluconate and zinc acetate; zinc glycinate and zinc lactate; zinc lactate and zinc acetate. In addition, zinc citrate trihydrate and zinc picolinate could be added to the mixture, as previously indicated. In all the cases, zinc sulfate mono hydrate could be added at a concentration below 1.5% in the finished product.

Viscosity

As mentioned, a high viscosity dipping (over 10 cP) is not desirable for spraying (carousel-type milking processes). Whereas if the viscosity is close to 1 cP, the sprayed product forms a ring and does not completely wet the udders when sprayed.

Therefore, to fully wet the udders and obtain good atomization, the viscosity of the dipping should be in the range of 3 to 5 centipoise. To achieve the proper viscosity, the humectant agent can be combined with an appropriate amount of thickening agent. The viscosity of the product is achieved through the correct mixture of glycerin, propylene glycol and PVA, by forming an emulsion with the inorganic phase that corresponds mainly to water, the aqueous solution of zinc salts.

The use of the combination of glycerin and PVA responds to the fact that, although glycerin contributes to the viscosity, it was found that increasing the content in the dipping to 20% did not achieve the target viscosity Similar tests with just PVA also did not show good results, as very small or very large amounts formed lumps in the dipping and the stability of the product was lost. However, by adjusting a mixture of glycerin and PVA with a weight ratio of 1.5 to 5, it is possible to obtain a stable formula, which meets the target viscosity, and exhibits good adherence to the cow's udders.

As indicated, the spray system of the robot limits the viscosity range. A product with a viscosity greater than 10 cP cannot be sprayed. In the range of 5-10 cP only a straight stream comes out, while in the range of 3-5 cP a wide spray is obtained that moistens the entire surface of the udders. Viscosity values under 3 cP, result in a concentric circle type irrigation that does not completely wet the udders, wetting the front of the udder and not the teats.

The spray system of the robot is programmed to operate for a period of 4-6 seconds in pre-dipping and post-dipping mode. This time is sufficient to spray 15-25 ml of the dipping per cow. Therefore, the challenge for a good dipping performance is that it must work at the robot's operating conditions and must be effective in controlling mastitis through a spray that soaks all the cow's udders, which is able to adhere and seal them, and that eliminates the bacteria present in the udders.

Biocide Effectiveness

The dipping having 10,000 ppm of zinc ions, from the mixture of zinc gluconate and zinc lactate (equivalent to 3.485% zinc gluconate and 1.865% zinc lactate) was subjected to quantitative germicidal rate tests (Time Kill Kinetics Assay, ASTM E2315). Briefly, 10 ml of the dipping were inoculated with a concentrated solution of bacteria in order to reach 10⁶ CFU/ml, then the mixture was agitated for 30 seconds, an aliquot was taken which was seeded by flooding on Plate Count agar, to then be incubated for 24 hours at 36° C. After this, the bacterial colonies were counted and the percentage of elimination was determined with respect to a control sample where the dipping was replaced by water, maintaining the test conditions described above.

The percentage of elimination of bacteria at 30 seconds contact time was: Escherichia coli (100%), Staphylococcus aureus (100%), Streptococcus uberis (99.99%), Streptococcus agalactiae (99.98%), Streptococcus dysgalactiae (99.90%), Staphylococcus coagulasa negative (100%), and Pseudomonas spp (99.99%).

For the qualitative evaluation of the bactericidal activity of the different dipping (salt mixtures) the Kirby Bauer method (Halo Method) was used, where a plate with Mueller Hinton agar was inoculated with a bacterial concentration of 1.5×10⁸ CFU/ml, to then, by punching out 4 holes of 5 mm diameter per 90 mm diameter plate. Each hole was independently inoculated with 35 microliters of dipping, and then the plates were incubated for 18-20 hours at 36° C. Finally, the diameter of the halo is measured. The larger the diameter, the greater the antibacterial activity.

Non-Corrosiveness

The fact of having a non-corrosive formulation allows the present invention to be a versatile composition, which can be used in milking robots, not only as a pre-dipping but also as a post-dipping, without changing the concentration of biocide organic zinc salts nor the viscosity or any other property of the formulation. The versatility of the invention also allows for use as pre- or post-dipping in other milking systems, such as the carousel type.

It is desirable to incorporate colorant or dye in the dipping solution. This way, it is easy to detect if the post dipping was applied or not and the quality of application. We have selected the mixture of blue and green food grade colorant. The concentration of colorant in the dipping is ideally 0.001%-0.003% for blue and 0.001%-0.003% for green. Not exceeding 0.004% in total. We have found that greater concentrations of colorant stain the visor of the camera of the robot, increasing the difficulty of locating the udder of the cow.

The amount of colorant in the present invention is almost 10 times less than those described in other dipping patents.

### EXAMPLES

The following examples are meant to illustrate the invention, but in no case to limit it.

### Example 1

One Kilogram of product (dipping) with a composition according to the present invention is made dissolving 8.362% of zinc gluconate in 80.838% of water, while mixing at 300 rpm at room temperature. After dissolution is completed, 6.5% of glycerin, 0.8% propylene glycol and 3.5% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.2 cP, measured with a Digital Viscometer, Model NDJ-1S at 25° C.

### Example 2

One Kilogram of product (dipping) with a composition according to the present invention is made dissolving 4.471% of zinc lactate in 84.729% of water, while mixing at 300 rpm at room temperature. After dissolution is completed, 6.5% of glycerin, 0.8% propylene glycol and 3.5% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.1 cP, measured with a Digital Viscometer, Model NDJ-1S at 25° C.

### Example 3

One Kilogram of product (dipping) with a composition according to the present invention is made dissolving 3.368% of zinc acetate in 85.832% of water, while mixing at 300 rpm at room temperature. After dissolution is completed 6.5% of glycerin, 0.8% propylene glycol and 3.5% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.2 cP, measured with a Digital Viscometer, Model NDJ-1S at 25° C.

The diameter of the halos was measured for the following bacteria:

TABLE 1
Diameter of Diffusion Inhibition Halo (mm) for 12.000 ppm of zinc ions, using different zinc
salts

| | Example 1 | Example 2 | Example 3 |
|---|---|---|---|
| Zinc salt | Zinc Gluconate | Zinc Lactate | Zinc Acetate |
| ppm Zinc | 12.000 | 12.000 | 12.000 |
| Staphylococcus aureus methicillin sensitive | 20 | 18 | 19 |
| ATCC 25923 | | | |
| Escherichia coli ATCC 25922 | 16 | 17 | 16 |
| Staphylococcus aureus - Isolated from | 20 | 20 | 20 |
| Clinical Mastitis | | | |
| Streptococcus agalactiae - Isolated from | 17 | 17 | 16 |
| Clinical Mastitis | | | |
| Escherichia coli (coliforme) - Isolated from | 16 | 16 | 16 |
| Subclinical Mastitis | | | |
| Streptococcus uberis - Isolated from | 18 | 17 | 18 |
| Subclinical Mastitis | | | |
| Streptococcus agalactiae - Isolated from | 18 | 18 | 17 |
| Subclinical Mastitis | | | |

The following examples (4 to 7) use a mixture of two zinc salts. Each zinc salt contributes 6,000 ppm of zinc ion.

### Example 4

One Kilogram of product (dipping) with a composition according to the present invention is obtained dissolving 4.181% of zinc gluconate, 2.235% of zinc lactate in 82.783% of water, while mixing at 300 rpm at room temperature. After dissolution is completed 6.5% of glycerin, 0.8% propylene glycol and 3.5% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.2 cP, measured with a Digital Viscometer, Model NDJ-1S at 25° C.

### Example 5

One Kilogram of product (dipping) with a composition according to the present invention is obtained dissolving 4.181% of zinc gluconate, 1.684% of zinc acetate in 83.335% of water, while mixing at 300 rpm at room temperature. After dissolution is completed 6.5% of glycerin, 0.8% propylene glycol and 3.5% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.2 cP, measured with a Digital Viscometer, Model NDJ-1S at 25° C.

### Example 6

One Kilogram of product (dipping) with a composition according to the present invention is obtained dissolving 2.235% of zinc lactate, 1.684% of zinc acetate in 85.281% of water, while mixing at 300 rpm at room temperature. After dissolution is completed 6.5% of glycerin, 0.8% propylene glycol and 3.5% of PVA, is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.2 cP, measured with a Digital Viscometer, Model NDJ-1S at 25° C.

### Example 7

One Kilogram of product (dipping) with a composition according to the present invention is obtained dissolving 1.967% of zinc glycinate, 3.371% of zinc lysinate in 83.861% of water, while mixing at 300 rpm at room temperature. After dissolution is completed 6.5% of glycerin, 0.8% propylene glycol and 3.5% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.2 cP, measured with a Digital Viscometer, Model NDJ-1S at 25° C.

TABLE 2
Diameter of Diffusion Inhibition Halo (mm) obtained with 12.000 ppm of zinc ions from a
mixture of two zinc salts

| | Example 4 | Example 5 | Example 6 | Example 7 |
|---|---|---|---|---|
| Zinc salt | Zinc | Zinc | Zinc Lactate | Zinc Glycinate |
| | Gluconate | Gluconate | Zinc | Zinc Lysinate |
| | Zinc Lactate | Zinc Acetate | Glycinate | |
| ppm Zinc | 12.000 | 12.000 | 12.000 | 12.000 |
| Staphylococcus aureus methicillin | 23 | 21 | 22 | 23 |
| sensitive ATCC 25923 | | | | |
| Escherichia coli ATCC 25922 | 19 | 20 | 19 | 21 |
| Staphylococcus aureus - Isolated from | 25 | 23 | 24 | 24 |
| Clinical Mastitis | | | | |
| Streptococcus agalactiae - Isolated | 20 | 21 | 20 | 20 |
| from Clinical Mastitis | | | | |
| Escherichia coli (coliforme) - Isolated | 20 | 19 | 21 | 20 |
| from Subclinical Mastitis | | | | |
| Streptococcus uberis - Isolated from | 21 | 20 | 21 | 21 |
| Subclinical Mastitis | | | | |
| Streptococcus agalactiae - Isolated | 21 | 21 | 20 | 21 |
| from Subclinical Mastitis | | | | |

Examples 8 to 10 show the results of Halo Inhibition measured for a mixture of two organic zinc salts, to which 1.098% of zinc sulfate monohydrate (a contribution of 4,000 ppm zinc ion) has been added. The total zinc ion concentration in the test is 28,000 ppm. The difference in zinc ion concentration was contributed in equal parts by the other salts, 12,000 ppm of zinc ion, each.

### Example 8

One Kilogram of product (dipping) with a composition according to the present invention is obtained dissolving 8.362% of zinc gluconate, 4.471% of zinc lactate, 1.098% zinc sulfate monohydrate in 69.069% of water, while mixing at 300 rpm at room temperature. After dissolution is completed 12% of glycerin, 1% propylene glycol and 4% of PVA it is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.9 cP, measured with a Digital Viscometer, Model NDJ-1S at 25° C.

### Example 9

One Kilogram of product (dipping) with a composition according to the present invention is obtained dissolving 4.471% of zinc lactate, 3.368% of zinc acetate, 1.098% zinc sulfate monohydrate in 74.063% of water, while mixing at 300 rpm at room temperature. After dissolution is completed 12% of glycerin, 1% propylene glycol and 4% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.8 cP, measured with a Digital Viscometer, Model NDJ-1S at 25° C.

### Example 10

One Kilogram of product (dipping) with a composition according to the present invention is obtained dissolving 3.934% of zinc glycinate, 6.742% of zinc lysinate, 1.098% zinc sulfate monohydrate in 71.226% of water, while mixing at 300 rpm at room temperature. After dissolution is completed 12% of glycerin, 1% propylene glycol and 4.0% of PVA is added, and mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

The viscosity of the dipping is 3.9 cP, measured with a Digital Viscometer, Model NDJ-1S at 25° C.

TABLE 3
Inhibition Halo Diameter (mm) obtained with 28,000 ppm of zinc ions from a mixture of two
organic zinc salts plus zinc sulfate monohydrate.

| | Example 8 | Example 9 | Example 10 |
|---|---|---|---|
| Zinc salt | Zinc Gluconate | Zinc Lactate Zinc | Zinc Glycinate Zinc |
| | Zinc Lactate | Acetate | Lysinate Zinc |
| | Zinc Sulfate | Zinc Sulfate | Sulfate |
| | Monohydrate | Monohydrate | Monohydrate |
| ppm Zinc | 28.000 | 28.000 | 28.000 |
| Staphylococcus aureus | 29 | 28 | 29 |
| methicillin sensitive ATCC | | | |
| 25923 | | | |
| Escherichia coli ATCC 25922 | 26 | 27 | 26 |
| Staphylococcus aureus - Isolated | 32 | 31 | 32 |
| from Clinical Mastitis | | | |
| Streptococcus agalactiae - | 26 | 26 | 27 |
| Isolated from Clinical Mastitis | | | |
| Escherichia coli (coliforme) - | 25 | 26 | 26 |
| Isolated from Subclinical Mastitis | | | |
| Streptococcus uberis - Isolated | 27 | 26 | 27 |
| from Subclinical Mastitis | | | |
| Streptococcus agalactiae - | 26 | 26 | 26 |
| Isolated from Subclinical Mastitis | | | |

Corrosive Effect

The corrosive effect of dipping was determined in different metallic and non-metallic parts, such as: iron, zinc-plated iron, 316 stainless steel and computer boards. Each of the pieces was placed independently in a 1 liter glass beaker containing 800 ml of dipping. The vessels were kept at 25° C. for six months, each piece being inspected visually and microscopically every two weeks for the first two months and once a month for the next 4 months. Inspections look for signs of attack on the surface, such as pitting, cracks, crevices, or color changes No signs of corrosion were observed on the parts. The dipping used is that of the examples.

Skin

An experimental test was designed to evaluate the quality of the skin and the tip of the udders, comparing them after the application of different pre and post dipping according to the following:

(a) Pre and post dipping according to the present invention (20,000 ppm of zinc ion from equal parts of zinc gluconate and zinc lactate); versus

(b) Pre and post dipping from a commercial formula based on 3% glycolic acid; and

(c) Pre and post dipping of a commercial formula based on 0.5% hydrogen peroxide and 1.7% lactic acid.

The test lasted 1 month and the evaluation panel included 100 cows per dipping, where each cow was milked three times a day, evaluating the skin quality of the 4 udders daily, marking a score for the skin of the teat and the tip of the teat according to the scale used in the standard evaluations of the industry.

TABLE 4
Udder Skin Quality Score after pre and post dipping application:
Summary of Results

| Condition of | Zinc Salts | Glycolic acid | Hydrogen peroxide + lactic acid |
|---|---|---|---|
| the skin | (a) | (b) | (c) |
| Normal | 100% | 60% | 20% |
| Dry | 0% | 20% | 25% |
| Cracked | 0% | 10% | 5% |
| Irritated | 0% | 10% | 25% |
| Flaked | 0% | 0% | 25% |

The score was assigned according to the standard evaluation in the industry, Teat End conditions (QCW-7: Teat End Conditions Scorecard).

TABLE 5
Evaluation of the tip of the teat after the application of different pre- and post dipping:
Summary of Results.

| | | | Hydrogen peroxide + lactic acid |
|---|---|---|---|
| Condition of the Tip | Zinc Salts (a) | Glycolic acid (b) | (c) |
| No ring | 90% | 50% | 30% |
| Soft ring | 10% | 35% | 40% |
| Wrinkled ring | 0% | 10% | 15% |
| Very Wrinkled ring | 0% | 5% | 15% |

The ring is formed by hyperkeratosis of the tip of the teat. The absence of a ring indicates a healthy condition.

Results show that zinc salts provide greater protection for teat tips and maintain udders in better conditions than commercial formulas based on different biocidal agents.

Examples 11-14: Comparison of the antibacterial activity of the invention versus commercial formulas based on different biocidal agents.

The antibacterial activity of the dipping composition according to the present invention was evaluated and compared with other commercial formulas based on different biocidal agents, in the same matrix solution. Antibacterial activity tests compare the diameters of the inhibition halos.

### Example 11

One Kilogram of product (dipping) is obtained dissolving 2.787% of zinc gluconate, 1.49% of zinc lactate, 1.098% of zinc sulfate monohydrate. Each zinc salt contributes with 4,000 ppm of zinc to the solution for a total of 12,000 ppm zinc. An 83.825% of water, while mixing at 300 rpm at room temperature is added. After total dissolution is achieved, 6.5% of glycerin, 0.8% propylene glycol and 3.5% of PVA are added and the composition is mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

### Example 12

One Kilogram of product (dipping) is obtained dissolving 1.922% of zinc sulfate monohydrate (a total of 7,000 ppm of zinc ion) and copper sulfate pentahydrate 2.75% (a total of 7,000 ppm of copper ion). An 84.528% of water, while mixing at 300 rpm at room temperature is added. After total dissolution is achieved, 6.5% of glycerin, 0.8% propylene glycol and 3.5% of PVA are added and the composition is mixed for 10 minutes at 500 rpm.

A volume of 35 microliters is added to the 5 mm diameter hole previously made in a Mueller Hinton plate inoculated with bacteria at a concentration of 1.5×10⁸ UFC/ml. The diameter of diffusion inhibition halo is measured after 20 hours incubation at 36° C.

### Example 13

One Kilogram of product (dipping) is obtained dissolving 3% glycolic acid (a total of 30,000 ppm of glycolic acid) and 86.2% of water, at 300 rpm at room temperature. After the mixing is completed, 6.5% of glycerin, 0.8% propylene glycol and 3.5% of PVA are added and the composition is mixed for 10 minutes at 500 rpm.

A volume of 35 microliters is added to the 5 mm diameter hole previously made in a Mueller Hinton plate inoculated with bacteria at a concentration of 1.5×108 UFC/ml. The diameter of diffusion inhibition halo is measured after 20 hours incubation at 36° C.

### Example 14

One Kilogram of product (dipping) is obtained mixing 0.5% hydrogen peroxide, 1.7% lactic acid, and 87% of water, at 300 rpm at room temperature. After the mixing is completed, 6.5% of glycerin, 0.8% propylene glycol and 3.5% of PVA are added and the composition is mixed for 10 minutes at 500 rpm.

A volume of 35 microliters is added to the 5 mm diameter hole previously made in a Mueller Hinton plate inoculated with bacteria at a concentration of 1.5×108 UFC/ml. The diameter of diffusion inhibition halo is measured after 20 hours incubation at 36° C.

The results of the diameters of the inhibition halos (mm) are shown in the following table.

TABLE 6
Diameters of the Inhibition Halos (mm) obtained from the invention versus formulas based on
the use of different biocidal actives

| | Example 11 | | | |
|---|---|---|---|---|
| | Zinc | | | Example 14 |
| | Gluconate | Example 12 | | Hydrogen |
| | Zinc Lactate | Copper Sulfate | Example 13 | peroxide + |
| | Zinc Sulfate | Zinc Sulfate | Glycolic Acid | lactic acid |
| Staphylococcus aureus methicillin | 23 | 8 | 10 | 20 |
| sensitive ATCC 25923 | | | | |
| Escherichia coli ATCC 25922 | 19 | 10 | 10 | 16 |
| Staphylococcus aureus - Isolated from | 25 | 10 | 11 | 20 |
| Clinical Mastitis | | | | |
| Streptococcus agalactiae - Isolated | 20 | 8 | 12 | 18 |
| from Clinical Mastitis | | | | |
| Escherichia coli (coliforme) - Isolated | 20 | 9 | 10 | 16 |
| from Subclinical Mastitis | | | | |
| Streptococcus uberis - Isolated from | 21 | 11 | 12 | 18 |
| Subclinical Mastitis | | | | |
| Streptococcus agalactiae - Isolated | 21 | 9 | 12 | 19 |
| from Subclinical Mastitis | | | | |

The results show that this invention, having 12,000 ppm of zinc ions (mixture of three zinc salts) has a higher antibacterial activity than (a) a mixture of 7,000 ppm of zinc ion plus 7,000 ppm of copper ion; (b) 3% glycolic acid; and (c) a mixture of 0.5% hydrogen peroxide and 1.7% lactic acid.

### Example 15

The dipping (solution) is prepared in an agitated tank equipped with an axial agitator with variable speed up to 700 rpm and which consists of the following steps:
a) A 1.5 cubic meter tank is loaded to prepare 1,000 kilograms of the present invention, adding between 770-810 kilograms of demineralized water.
b) Zinc salts are added and the system is agitated from 15 minutes at 400 rpm.
c) Glycerin and propylene glycol are added in a ratio from 1.5-5 by weight and with agitation for 15 minutes at 600 rpm.
d) PVA is added in the range 2% to 4%.
e) Agitation is continued 5 minutes at 600 rpm.
f) Viscosity is measured and the absence of lumps is visually inspected.
g) An aqueous solution of dye previously dissolved in water is added so that the concentration of the blue pigment is in the order of 0.001%-0.003% and of the green pigment in the same range. The sum of the pigments must not exceed 0.004%.

1. A composition for the treatment of mastitis, wherein it comprises:
a) at least one water soluble organic zinc salt selected for having a functional group similar to proteins with biocide properties at a concentration of 5,000 to 60,000 ppm of zinc ion;
b) humectant agents compatible with foods;
c) a viscosity agent (viscosity control agent) such that combined with humectant agents allows a target viscosity of 3 to 5 cP to be achieved; and
d) demineralized water up to 100%,
where the composition is non corrosive and eliminates over 99.9% of bacteria in less than 30 seconds of contact time.

2. The composition according to claim 1, wherein the water soluble organic zinc salt is selected from the group consisting of zinc gluconate, zinc glycinate, zinc lactate, zinc citrate trihydrate, zinc picolinate, zinc acetate, and zinc lysine.

3. The composition according to claim 2, wherein it preferably comprises a combination of two or more water soluble organic zinc salts.

4. The composition according to claim 3, wherein the preferred combination of zinc salts are zinc gluconate and zinc lactate in a ratio of 5/1 to 1/5 by weight, zinc gluconate and zinc glycinate in a ratio of 5/1 to 1/5 by weight, zinc gluconate and zinc acetate in a ratio of 5/1 to 1/3 by weight, zinc glycinate and zinc lactate in a ratio 3/1 to 1/3 by weight, zinc lactate and zinc acetate in a ratio of 5/1 to 1/5 by weight.

5. The composition according to claim 4, wherein the composition also comprises a third zinc salt such as zinc citrate trihydrate and/or zinc picolinate in a concentration of 1% and 0.5% by weight, respectively in the final product.

6. The composition according to claim 1, wherein the humectant agent is at a concentration between 5-20% by weight.

7. The composition according to claim 6, wherein that the humectant agents are selected from the group consisting of glycerin, propylene glycol, hexylene glycol, butylene glycol, glyceryl triacetate, aloe vera, pyrrolidone carboxylic acid, sorbitol, sodium lactate, and panthenol.

8. The composition according to claim 7, wherein the preferred humectant agent is a combination of glycerin and propylene glycol.

9. The composition according to claim 8, wherein the glycerin and propylene glycol are present in a ratio in the range of 6 to 1 to 15 to 1 by weight.

10. The composition according to claim 1, wherein the viscosity controlling agent is at a concentration range between 2-4% by weight.

11. The composition according to claim 10, wherein the preferred viscosity controlling agent is polyvinyl alcohol (PVA).

12. The composition according to claim 3, wherein one of the water soluble organic zinc salts is replaced by zinc sulfate.

13. The composition according to claim 12, wherein the preferred concentration of zinc sulfate monohydrate should be less than 1.5%, preferably less than 1.1%.

14. The composition according to claim 1, wherein it can optionally comprise additional agents selected from colorants, perfume, vitamins, alpha-hydroxy-acids, beta hydroxy-acids and hydroquinone.

15. The composition according to claim 1, wherein it is a sprayable composition for treating mastitis.

16. Use of a composition according to claim 1, wherein is used for preparing a formulation that prevents the formation of ring by hyperkeratosis of the udder tip.

17. Process for elaborating a composition for treating mastitis, wherein it comprises the following steps:
a) loading demineralized water into the tank (balance at 100% formula);
b) adding the soluble organic zinc salts to the tank and agitate the mixture for 10-20 minutes at 300-400 rpm until the complete dissolution of zinc salts is achieved, where all the salts are dissolved before adding the organic part in order to avoid the formation of lumps;
c) adding the humectant keeping the agitation 10-20 minutes at 300-600 rpm to ensure the formation of an emulsion;
d) adding the viscosity agents in the range 2% to 4% in order to achieve the target viscosity, defined as between 3 and 5 centipoise;
e) agitating for 5-10 minutes at 300-600 rpm until homogeneous mixture is achieved; and
f) measuring the viscosity and verifying the absence of lumps, then add coloring not exceeding a maximum concentration of 0.004%,
all the stages of the process are performed at room temperature.
